(19) United States
(12) Patent Application Publication
(10) Pub. No.: US 2007/0244724 A1
(43) Pub. Date: Oct. 18, 2007

(54) CASE BASED OUTCOME PREDICTION IN A REAL-TIME MONITORING SYSTEM

(76) Inventors: John W. Pendergast, Germantown, WI (US); Donald E. Brodnick, Cedarburg, WI (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/279,584

(22) Filed: Apr. 13, 2006

(51) Int. Cl. A61B 5/00

(52) U.S. Cl. 705/3; 600/300

(57) ABSTRACT

A method for providing real-time support in interpreting medical record data and monitoring parameters received from a patient in order to assess the possible outcomes and treatment history for the patient based upon prior patients having similar monitoring parameters and medical record data. The system includes a monitoring device that obtains monitoring parameters from the patient and communicates with a historic reference database that includes numerous historic, stored patient records. Upon receiving the monitoring parameters and medical record data for the patient, the historic reference database identifies one or more historic patient records that closely correspond to the patient being treated. Based upon the identified historic patient records, the physician is presented with a likely outcome and proposed course of treatment.

### BACKGROUND OF THE INVENTION

The present invention relates to a method and system for providing a case-based outcome prediction for a patient. More specifically, the present invention relates to a monitoring device that obtains real-time monitoring data from a patient and accesses a historic reference database to receive a predicted outcome for the patient based upon real-time monitoring data and other medical record data for the patient.

In the present day healthcare environment, a wide variety of monitoring data and medical record data is received for each patient as the patient is being monitored in a healthcare facility. This information can include medical record information relating to the patient's history, monitoring parameters obtained from the patient, waveform data from monitoring devices connected to the patient and various outcome data associated with the treatment of the patient within a healthcare facility. Since most patient records are stored electronically, this information is typically stored in a database that is accessed each time the patient is treated either in the healthcare facility or at another facility in communication with the healthcare facility. Although the historic database record is useful in treating the patient at a later date, information received during the treatment of prior patients is typically not utilized in analyzing and treating a current patient.

In addition, due to the advent of new and innovative monitoring techniques and measurement devices, a physician is typically presented with an almost overwhelming amount of information when viewing the patient's medical records. Due to the vast amount of real-time information and monitoring parameters presented to the physician at one time, it is often difficult for the physician to recognize certain medical conditions that the physician may not see on a regular basis. Alternatively, the physician may be presented with a patient having a medical condition that occurs very rarely and thus the condition may not be readily apparent to the physician upon initially viewing the monitoring parameters and patient's medical record data. However, the monitoring parameters and medical record data received from the patient may closely correspond to a patient previously treated either in the healthcare facility or a related facility in the close geographic area.

Since most patient records are stored within some type of electronic information database, it is desirable to use an automated process to access the historic database such that the monitoring parameters and medical record data received from the patient can be compared to historic patient records, such that treatment methods and predicted outcomes can be generated based upon the historic patient data. Further, it is desirable to utilize the historic patient records to not only predict an outcome for an individual patient, but to provide a summary of possible treatments that have been utilized for similar patients having monitoring parameters and medical record data corresponding to the patient currently being treated.

### SUMMARY OF THE INVENTION

The present invention provides real-time support in interpreting medical record data and monitoring parameters received from a patient in order to assess the possible outcomes and treatment history for the patient based upon prior patients having similar monitoring parameters and medical record data. The system includes a monitoring device that obtains monitoring parameters from the patient and communicates with a historic reference database that includes numerous historic, stored patient records. Upon receiving the monitoring parameters and medical record data from the monitoring device associated with the patient, the historic reference database compares the monitoring parameters and medical record data to the numerous stored patient records contained within the database.

It is contemplated that the historic reference database can exist over various different geographic areas. As an example, the reference database may include historic patient records from the healthcare facility in which the patient is being treated or the database may be a regional, national or global database that includes an extremely large number of historic patient records to which the information from the patient being treated can be compared. The reference database may be maintained by the healthcare facility or may be a subscription service provided by an organization and accessed by healthcare subscribers.

Once the historic database identifies one or a plurality of historic patient records that include similar monitoring parameters and medical record data to the patient being monitored, the historic database returns information relating to the outcome and treatment of the historic patient or patients to either the monitoring device or some other device that can be accessed by the physician. The information received from the historic reference database may include outcome history, such as the length of hospital stay for the patient having similar characteristics, the mortality/morbidity rate for similar patients, as well as the level of acuity for similar type patients. In addition to outcome information, the historic reference database returns diagnosis information as well as treatment schedules followed for similar patients.

Based upon the outcome information, treatment information and diagnosis information, the physician can then more quickly and easily diagnose the patient, develop a treatment schedule and provide outcome information to the patient based upon the sampling of historic patients that had similar medical record data and monitoring parameters.

In addition to communicating the information to the treating physician, the historic reference database can also trend data in similar geographic areas, age populations or other relevant demographics and provide this trended information to a healthcare body, such as a regulatory agency or similar organization. Based upon the trended data, the healthcare body can provide warnings, alerts or other information to the general public.

### BRIEF DESCRIPTION OF THE DRAWINGS

The drawings illustrate the best mode presently contemplated of carrying out the invention. In the drawings:

FIG. 1 is a schematic illustration of the outcome prediction and real-time monitoring system of the present invention;

FIG. 2 is a schematic illustration of the historic reference database including multiple historic patient records and the communication between the database and the monitoring device; and

FIG. 3 is an illustration of a sample history patient record with the historic information stored therein.

### DETAILED DESCRIPTION OF THE INVENTION

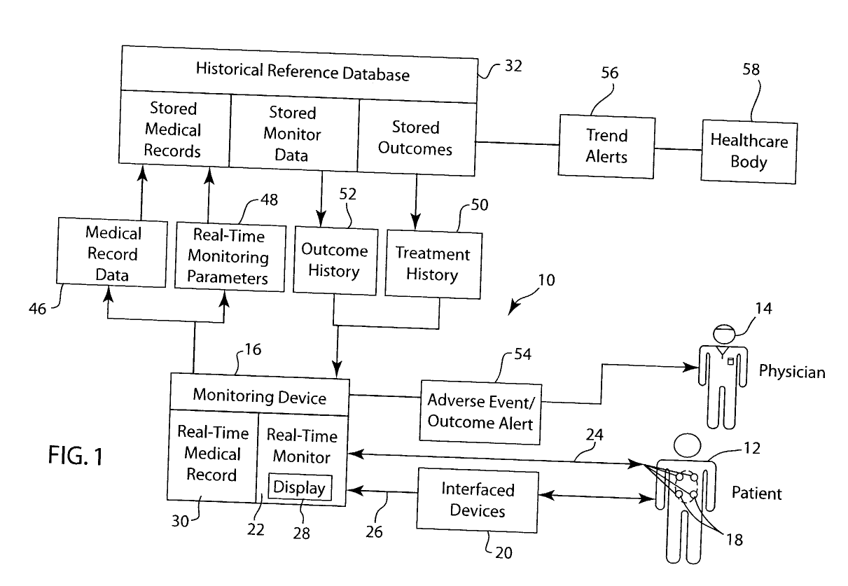

A system 10 for monitoring a patient 12 and predicting an outcome for the patient to be used by a physician 14 is generally shown in FIG. 1. The system 10 includes a monitoring device 16 that receives various monitoring parameters from the patient 12 either through a series of sensors 18 positioned on the patient or through various different interfaced devices 20 connected to the patient to receive various different types of monitoring parameters. In the embodiment of the invention shown in FIG. 1, the monitoring parameters received from the patient can include, but are not limited to, ECG data, heart rate, heart rate variability, T-wave alternans, QT intervals, ST levels, invasive and non-invasive blood pressures, systolic pressure variation, cardiac output, blood oxygen saturation, EEG, bispectral index, entropy, respiratory mechanic parameters such as airway flow, volume and pressure measures, respiratory rate, and inhaled and exhaled gas concentrations. The invention should not be limited to the examples described above and shown in the drawings, but should be interpreted to include any type of monitoring parameter that can be obtained from the patient through either currently available or yet to be produced patient monitoring systems. The monitoring parameters received from the patient 12 are delivered to the real-time monitor 22 along the communication line 24.

In addition to receiving the monitoring parameters directly from the patient 12, the real-time monitor 22 can receive additional patient data from the interface device 20. As an example, the interface device 20 may be an x-ray machine, a ventilator, an anesthesia machine, a continuous cardiac output monitor or any other type of device that obtains and provides physiological patient data for the patient. The interface device 20 communicates to the real-time monitor 22 over the communication line 26. In the embodiment of the invention shown in FIG. 1, the interface devices 20 are contemplated as having internal processing capabilities and are capable of carrying out independent diagnostic testing, providing medical treatments as well as delivering physiological data to the real-time monitor 22 over the communication line 26. The real-time monitor 22 includes a display 28 that allows the real-time monitor 22 to display relevant patient information to the treating physician 14.

As shown in FIG. 1, the monitoring device 16 includes a real-time medical record 30 for the patient 12 being treated by the physician. Although the real-time medical record 30 is shown as being included within the monitoring device 16, it should be understood that the real-time medical record most likely will be stored at a remote location, such as in a hospital information system (HIS), which can be accessed by the monitoring device 16 either through a hardwire connection or using wireless communication techniques. Typically, the real-time medical record 30 for the patient includes, but is not limited to, patient demographic data such as the patient's age, height, weight, family history and genetics, disease states, prior diagnoses, caregiver documentation, drugs taken or given, medical and surgical procedures completed, laboratory and imaging results, as well as other diagnostic test results. This type of information is typically stored in a patient's electronic medical record and is accessed by a physician treating the patient either directly through the monitoring device 16 or through computer terminals accessible throughout the hospital or healthcare facility and in communication with the electronic database that stores the medical records for the patient.

During the typical treatment of the patient, the physician 14 views the monitoring data on the display 28 for the patient as well as accesses the real-time medical record for the patient on the monitoring device. Based upon the monitoring parameters and medical record data for the patient, the physician uses his or her knowledge and experience to estimate a predicted outcome for the patient and develop a course of treatment based upon the diagnosis of the treating physician. In most cases, the outcome prediction and treatment schedule determined by the physician provides a very high level of treatment for the patient. However, the large variety of monitoring parameters and medical record data available for the patient 12 presents the physician 14 with an often overwhelming volume of information that is difficult to decipher in coming up with a treatment strategy. Further, if the patient 12 is suffering from a unique, rarely seen malady, or multiple diseases or injuries, the physician 14 may not immediately recognize the symptoms, which may result in either an improper or non-optimal treatment.

Referring back to FIG. 1, the system of the present invention utilizes a historic reference database 32 that can be accessed by the monitoring device 16 using conventional communication techniques, such as over the internet or a direct wired connection. The historic reference database 32 may be located within the healthcare facility in which the patient 12 and physician 14 are located. However, to provide maximum benefit for the system 10, it is contemplated that the historic reference database 32 may be a regional, national or global database that is accessible by the monitoring device 16 in real-time. It is contemplated that the historic reference database 32 may be publicly accessible or may be hosted by a commercial provider and accessed by the monitoring device on a subscription basis.

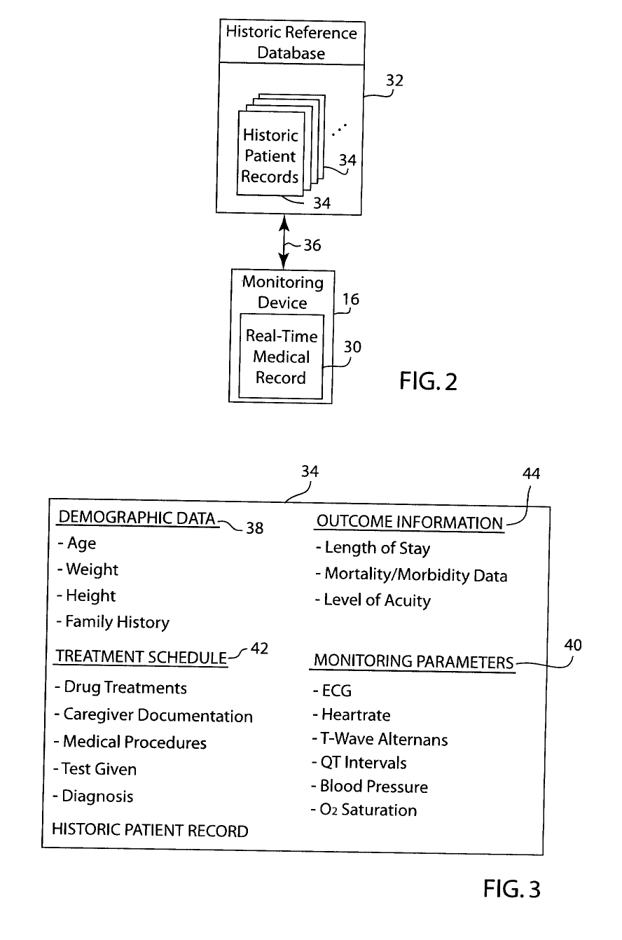

As shown in FIG. 2, the historical reference database includes a series of historic patient records 34 that can be accessed by the monitoring device 16 through the communication channel 36. The historic patient records are stored within the historic reference database 32 and include all of the medical record data, monitoring parameters, treatments and outcome information for a large number of patients. Preferably, each of the historic patient records 34 is stripped of patient identification information such that the historic patient records cannot be correlated to an actual patient.

Referring now to FIG. 3, thereshown is a representation of a historic patient record 34 stored within the historic reference database. As illustrated, the historic patient record 34 includes demographic data 38 for the patient, including but not limited to the age, weight, height and family history for the patient. The patient record 34 also includes monitoring parameters 40 taken from the patient during the course of the patient's history or treatment. The monitoring parameters 40 can include, but are not limited to, ECG signals, heart rate values, T-wave alternans, QT intervals, blood pressure and 02 saturation levels. Although several examples of the different types of the monitoring parameters 40 are shown and described in FIG. 3, it should be understood that various other monitoring parameters are capable of being stored within the historic patient record 34.

In addition to the demographic data 38 and the monitoring parameters 40, the historic patient record 34 includes a listing of the treatments utilized with the patient. These treatments may include the type of drugs taken by the patient, caregiver documentation regarding the patient treatment, medical procedures performed on the patient, tests given during the treatment as well as diagnosis information for the patient. Finally, the historic patient record includes outcome information 44 that provides information as to the ultimate outcome obtained for the patient based upon the treatment, monitoring parameters and medical record data. The outcome information 44 may include, but is not limited to, information related to the length of stay of the patient, the mortality/morbidity and the level of acuity in treating the patient.

Referring back to FIG. 2, since most patients being treated in modem health facilities are provided with an electronic patient record, the historic patient records 34 contained within the historic reference database provide a vast amount of information relating to the outcome and treatment for patients along with the medical record data and monitoring parameters for the individual patient. It is contemplated that the historic reference database 32 could include thousands, if not millions, of historic patient records 34 that can be accessed by the monitoring device 16 through the communication line 36.

Referring back to FIG. 1, the monitoring device 16 is in two-way communication with the historic reference database 32. The monitoring device 16 delivers medical record data 46, which may include demographic data, treatment schedule information and outcome information, and real-time monitoring parameters 48 for comparison to the historic patient records within the historic reference database 32. In the preferred embodiment of the invention, the medical record data 46 and real-time monitoring parameters 48 are continuously compared to and correlated with the historic patient records to find the closest historic patient record corresponding to the medical record data and real-time monitoring parameters received from the monitoring device 16. Since the historic reference database 32 may contain vast numbers of historic patient records, the real-time monitoring parameters 48 and medical record data 46 for the patient being treated will most likely closely correspond to at least one other historic patient record 34 contained within the historic reference database.

Once the historic reference database identifies one or more historic patient records that closely correspond to the real-time monitoring parameters 48 and medical record data 46 received from the monitoring device 16, the historic reference database 32 communicates the treatment history and outcome history from the identified historic patient records to the monitoring device 16. If the historic reference database 32 identifies more than one historic patient record that closely corresponds to the medical record data 46 and the real-time monitoring parameters 48 from the monitoring device 16, the historic reference database 32 will correlate the information and provide compiled information relating to the outcome history 52 and treatment history 50 to the monitoring device 16. Alternatively, the historic reference database 32 can provide multiple historic patient records to the monitoring device 16, each of which can then be reviewed by the physician 14. As an example, if the historic reference database 32 identifies three historic patient records that closely correspond to the real-time monitoring parameters and medical record data for the patient 12, the physician 14 can review the three historic patient records and determine which course of treatment resulted in the most positive outcome for a similar patient. In this manner, the physician 14 is presented with similar historic patient records to the patient he is currently treating and can assess the type of treatment and outcome associated with patients having medical record data and real-time monitoring parameters that closely correspond to the patient currently being treated.

In the embodiment of the invention shown in FIG. 1, the historic patient record is shown on the display 28 of the real-time monitor 22. However, if the monitoring device 16 receives an alert relating to an adverse event or adverse possible outcome, this information packet can be immediately delivered to the physician 14, such as through a pager, PDA or other type of alerting device. As an example, if the real-time monitoring parameters received from the patient begin to change such that the historic reference database 32 identifies a significant immediate risk to the patient, the monitoring device 16 can generate the information packet alert 54 to the physician indicating the worsening of the patient's condition. Once again, the monitoring device 16 is able to relay the real-time monitoring parameters 48 to the historic reference database 32, where the real-time monitoring parameters 48 can be compared to historic patient records.

In the preferred embodiment of the invention, the historic reference database 32 includes various different database algorithms that allow the database 32 to compare the real-time monitoring parameters 48 and medical record data 46 to similar information included in each of the historic patient records 34. Based upon a ranking and matching system, the historic reference database 32 can identify either one or more of the historic patient records 34 that closely correspond to the real-time monitoring parameters and medical record data received from the patient. Optionally, depending upon the condition which the patient 12 is being treated for, the monitoring device 16 can selectively supply only the most relevant real-time monitoring parameters 48 and medical record data 46 to the historic reference database 32 to insure that the historic reference database 32 identifies the historic patient records of greatest interest.

As discussed, once the most relevant record or records have been identified, the outcome history 52 and the treatment history 50 for each of the records are relayed back to the monitoring device 16. In this manner, the interaction between the monitoring device 16 and the historic reference database 32 allows for the identification of historic patient records that closely correspond to the information received from the patient 12 in real-time. Thus, the system 10 of the present invention allows the physician to be presented with outcome history and treatment history for patients having similar characteristics to the patient currently being treated. Further, this information in presented to the physician in real-time, since the monitoring device 16 is typically placed close to the patient 12 to receive the monitoring information in real-time.

In addition to alerting the physician of adverse events and potential outcomes, the historic reference database 32 can be configured to generate trend alerts 56 to various different healthcare agencies. As an example, if the historic reference database 32 is collecting numerous related historic patient records that indicate an outbreak of a certain medical condition within a concentrated geographic area, the historic reference database 32 may generate a trend alert 56 to a healthcare body, such as the CDC. Since the historic reference database 32 may be collecting patient records either on a regional or national scale, the historic reference database 32 is in a unique position to identify trends occurring over a relatively large geographic area, which was currently unavailable.

Once the physician 14 has completed the treatment on the patient 12, the monitoring device 16 will remove information that could identify the patient, and will upload the complete patient record into the historic reference database 32 such that the treatment procedures and outcome for the patient can be utilized in the future for other patients. In this manner, the historic reference database 32 continues to grow and provide a larger database of patient records that can be accessed and utilized in generating predicted outcomes and treatment schedules.

As can be understood by the above description, the clinical practice of the physician 14 can be assessed real-time against a historical perspective, improving real-time clinical decision making that allows for adjusted or early intervention and improved patient outcomes, ultimately saving time, money and effort for the clinician, the institution and healthcare financing/reimbursement bodies while simultaneously improving patient care and patient quality of life. The continued use of the system of the invention produces an ever improving benefit over time through the growth of the historic reference database and the addition of new case files containing the outcome of emerging treatment protocol. As stated, the historic reference database 32 can be used to monitor local, regional, national or global health for epidemiological or security purposes.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

We claim:

1. A method of monitoring a patient, comprising the steps of:
acquiring monitoring parameters from the patient;
acquiring medical record data for the patient;
accessing a historic reference database including a plurality of historic patient records each having historic monitoring parameters, historic medical record data and a stored outcome;
identifying at least one historic patient record that closely corresponds to the monitoring parameters and medical record data from the patient; and
displaying the stored outcome for the identified historic patient record as a predicted outcome for the patient.

2. The method of claim 1 further comprising the steps of storing the monitoring parameters and the medical record data from the patient in the historic reference database such that the stored monitoring parameters and the stored medical record data can be later searched as part of the historic reference database.

3. The method of claim 2 further comprising the step of removing patient identification information from the monitoring parameters and medical record data prior to storing the monitoring parameters and the medical record data.

4. The method of claim 1 further comprising the step of generating an alert to a treating physician based upon the predicted outcome for the patient.

5. The method of claim 1 further comprising the steps of:
identifying a plurality of historic patient records that closely correspond to the monitoring parameters and the medical record data for the patient; and
providing a probability of an outcome based upon the plurality of identified historic patient records.

6. The method of claim 1 wherein each of the historic patient records includes a treatment statement, wherein the treatment statement is displayed with the predicted outcome.

7. The method of claim 1 wherein the historic patient record that closely correspond to the monitoring parameters and the medical record data is identified in real-time with the acquisition of the monitoring parameters for the patient.

8. The method of claim 1 wherein the monitoring parameters are acquired by a monitoring device in close proximity to the patient and the predicted outcome is displayed on the monitoring device.

9. The method of claim 8 wherein the predicted outcome includes mortality rates.

10. The method of claim 8 wherein the demographic data from the patient is acquired from an electronic medical record accessible by the monitoring device.

11. A method of predicting an outcome and generating a treatment procedure for a patient, the method comprising the steps of:
acquiring monitoring parameters from the patient at a monitoring device located proximate to the patient;
acquiring medical record data for the patient;
accessing a historic reference database including a plurality of stored historic patient records, each historic patient record having historic monitoring parameters, historic medical record data and a stored outcome;
identifying at least one historic patient record that closely corresponds to the monitoring parameters and the medical record area obtained from the patient; and
displaying the stored outcome for the identified historic patient record as a predicted outcome for the patient.

12. The method of claim 1 1 further comprising the step of generating an alert to a treating physician based on the predicted outcome.

13. The method of claim 12 wherein the alert is displayed on the monitoring device.

14. The method of claim 1 1 wherein each of the historic patient records includes a treatment statement, wherein the treatment statement is displayed with the predicted outcome.

15. The method of claim 11 wherein the historic patient records that closely correspond to the monitoring parameters and the medical record data for the patient are identified in real-time with the acquisition of the monitoring parameters from the patient.

16. The method of claim 11 wherein the historic reference database is located remotely from the healthcare facility and includes historic patient records for patients other than those treated in the healthcare facility.

17. The method of claim 1 1 wherein each patient record of the historic reference database includes stored treatment schedules for each of the patients, wherein the stored treatment schedule for the identified patient record is displayed on the monitoring device.

18. A method of predicting an outcome and generating a treatment procedure for a patient, the method comprising the steps of:
acquiring monitor parameters from the patient at a monitoring device located proximate to the patient;
acquiring medical record data for the patient;
accessing a historic reference database including a plurality of stored historic patient records, each historic patient record having historic monitoring parameters, historic medical record data and a treatment outcome;
identifying a plurality of patient records that closely correspond to the monitoring parameters and the medical record data obtained from the patient;
predicting an outcome for the patient based upon the treatment outcomes in each of the identified historic patient records; and
displaying the predicted outcome for the patient.

19. The method of claim 18 wherein each of the historic patient records includes a treatment statement, wherein a proposed treatment statement is determined based upon the treatment statement of each of the identified historic patient records.

20. The method of claim 18 wherein the plurality of historic patient records are identified in real-time with the acquisition of the monitoring parameters from the patient.
